(19) United States
(12) Patent Application Publication
(10) Pub. No.: US 2018/0192779 A1
(43) Pub. Date: Jul. 12, 2018

(54) TV BED, TV, BED, AND METHOD FOR OPERATING THE SAME

(71) Applicant: BOE TECHNOLOGY GROUP CO., LTD., Beijing (CN)

(72) Inventors: Han Yan, Beijing (CN); Qi Zeng, Beijing (CN); Lihua Geng, Beijing (CN); Xiaoyu Li, Beijing (CN)

(73) Assignee: BOE TECHNOLOGY GROUP CO., LTD., Beijing (CN)

(21) Appl. No.: 15/741,754

(22) PCT Filed: Jan. 25, 2017
(86) PCT No.: PCT/CN2017/072552

§ 371 Date: Jan. 3, 2018

(30) Foreign Application Priority Data
May 30, 2016 (CN) 201610371674.1

(51) Int. Cl. A47C 20/04; A47C 21/00; A61B 5/00; G06K 9/00; H04N 21/442; H04N 21/443; H04N 21/41; H04N 21/4223

(52) U.S. Cl. CPC A47C 20/04; A47C 21/003; A61B 5/4809; G06K 9/00228; H04N 21/4223; H04N 21/44218; H04N 21/4436; H04N 21/4131; G06K 9/00315

(57) ABSTRACT

The present application discloses a TV bed, including: a bed and a TV. The bed comprises a bed body with a main position and a headboard portion, a bed sensing unit, a bed receiver, a bed sender and a first controlling unit; and the TV comprises a detecting unit, a determining unit, a generating unit, a TV receiver, a TV sender, and a second controlling unit, the bed receiver being configured to receive command signals from the TV sender, and the TV receiver being configured to receive command signals from the bed sender. The TV is configured to generate a bed-control command based on a user's face position in the bed and is configured to send the bed-control command to the bed; and the bed is configured to receive the bed-control command and is configured to control the headboard portion to move to a position based on the bed-control command.

### CROSS-REFERENCE TO RELATED APPLICATION

This application claims priority to Chinese Patent Application No. 201610371674.1, filed on May 30, 2016, the content of which is incorporated by reference in the entirety.

### TECHNICAL FIELD

The present invention relates to smart appliances, more particularly, to a TV bed, a TV, a bed, and a method for operating the TV bed.

### BACKGROUND

Users often lie in bed when watching TV in the bedroom. When watching TV, a user often uses a thick pillow behind his back or under his neck, and the thick pillow can cause discomfort. In addition, if the user falls asleep, using a thick pillow behind his back or under the neck may severely affect the health of the user's cervical vertebra and lumber vertebra.

In conventional technology, when a user is lying on his/her back in bed when watching TV, the user may change the viewing angle by controlling the TV to slide on a sliding rail. However, this viewing angle may not be a comfortable viewing angle and may cause the user to feel fatigue. Watching TV in an uncomfortable viewing angle for a long time may severely affect the health of the user's cervical vertebra and lumbar vertebra.

### SUMMARY

In one aspect, the present invention provides a TV bed, including: a bed and a TV, wherein the bed comprises a bed body with a main portion and a headboard portion, a bed sensing unit, a bed receiver, a bed sender and a first controlling unit; and the TV comprises a detecting unit, a determining unit, a generating unit, a TV receiver, a TV sender, and a second controlling unit, the bed receiver being configured to receive command signals from the TV sender, and the TV receiver being configured to receive command signals from the bed sender. The TV is configured to generate a bed-control command based on a user's face position in the bed and is configured to send the bed-control command to the bed; and the bed is configured to receive the bed-control command and is configured to control the headboard portion to move to a position based on the bed-control command.

Optionally, the TV is configured to determine whether the user is in a fatigue state, a sleeping state, or a wake-up state and is configured to generate a bed-control command and a TV command according to a result of determination; the bed is configured to receive the bed control-command and is configured to control the headboard portion according to the bed-control command; and the TV is configured to adjust TV parameters according to the TV command.

Optionally, the TV is configured to determine whether the user is in the fatigue state, the sleeping state, or the wake-up state by scanning the user's face.

Optionally, the bed is configured to measure user data and sends the user data to the TV; the TV is configured to determine whether the user is in the fatigue state, the sleeping state, or the wake-up state based on the user data.

Another aspect of the present disclosure provides a TV bed, including: a bed and a TV, wherein the bed is configured to comprise a bed body with a main portion and a headboard portion connected to the main portion, a receiver, and a first controlling unit; and the TV comprises a generating unit and a sender, the receiver. The generating unit is configured to generate a bed-control command based on a user's face position in the bed; the sender is configured to send the bed-control command to the receiver of the bed; the receiver is configured to receive the bed-control command; and the first controlling unit is configured to control the headboard portion to move to a position based on the bed-control command.

Optionally, the bed further including a storage unit, the TV further including a detecting unit and a first determining unit. The storage unit is configured to store parameters of the position; the detecting unit is configured to detect a position of a user's face; the first determining unit is configured to determine when the position of the user's face is in a range, and is configured to trigger the generating unit to generate the bed-control command when the position of the user's face is determined to be in the range, the bed-control command comprising an ascend command such that the first controlling unit retrieves the position from the storage unit to move the headboard portion to the position based on the ascend command.

Optionally, the first determining unit is configured to trigger the detecting unit to continue to check the position of the user's face when the position of the user's face is determined to be out of the range.

Optionally, the bed further comprising a bed-control terminal. The bed-control terminal is configured to accept a terminal command comprising an adjusted position from the user and is configured to send the terminal command to the receiver of the bed such that the first controlling unit retrieves the adjusted position from the terminal command and controls the headboard portion to move to the adjusted position.

Optionally, the first controlling unit is configured to output the adjusted position to the storage unit; and the storage unit is configured to set parameters of the adjusted position to be parameters of the position.

Optionally, the TV further includes a second determining unit and a detecting unit. The detecting unit is configured to detect a user's facial state; and the second determining unit is configured determine when the user is in a fatigue state or a sleeping state, and is configured to trigger the generating unit to generate the bed-control command when the user is in the sleeping state, the bed-control command comprising a reset command such that the first controlling unit controls the headboard portion to move to a horizontal position based on the reset command, the reset command comprising an ascend-descend speed such that the first controlling unit controls the headboard portion to move according to the ascend-descend speed.

Optionally, the TV further includes a second controlling unit, wherein the second controlling unit is configured to: turn off the TV based on a power-off command generated by the generating unit when the second determining unit determines the user is in the sleeping state; and decrease values of TV parameters based on a TV command generated by the generating unit when the second determining unit determines the user is in the fatigue state.

Optionally, the second determining unit is configured to determine when the user is in the fatigue state or sleeping state according to one or more fatigue detection algorithms, the one or more fatigue detection algorithms including an algorithm based on the user's eye closing time in a time period.

Optionally, the bed further includes a first connecting portion. The main portion and the headboard portion are configured to be connected through the first connecting portion; and the headboard portion, following the bed-control command, is configured to move about the first connecting portion to reach the position.

Optionally, the bed further includes a plurality of sub-headboard portions and a plurality of second connecting portions arranged in parallel with the first connecting portion, wherein the plurality of sub-headboard portions, following the bed, are configured to move about one or more second connecting portions until a portion of the headboard portion reaches the position.

Optionally, the bed further includes a sensing unit and a sender and the TV further comprising a receiver. The sensing unit is configured to collect user movement data; the sender of the bed is configured to send the user movement data to the receiver of the TV; and the generating unit is configured to generate a bed-control command and a TV command based on the received user movement data.

Optionally, the sender of the TV is configured to send the bed-control command to the bed; the receiver is configured to receive the bed-control command; the first controlling unit is configured to adjust the headboard portion according to the bed-control command; and the second controlling unit is configured to adjust the TV parameters according to the TV command.

Another aspect of the present disclosure provides a method for controlling a TV bed, the TV bed including a bed and a TV, wherein the bed comprises a bed body, a receiver, and a first controlling unit; and the TV comprises a generating unit and a sender, the bed body comprising a main portion and a headboard portion connected to the main portion, the method including: generating, by the generating unit, a bed-control command based on a user's face position in the bed; sending, by the sender of the TV, the bed-control command to the receiver of the bed; receiving, by the receiver of the bed, the bed-control command; and controlling, by the first controlling unit, the headboard portion of the bed body to move to a position based on the bed-control command.

Optionally, the bed further comprising a storage unit, the TV further comprising a detecting unit and a first determining unit, wherein a process before generating the bed-control command includes: storing, by the storage unit, parameters of the position; detecting, by the detecting unit, a position of a user's face; determining, by the first determining unit, when the position of the user's face is in a range, and triggering, by the first determining unit, the generating unit to generate the bed-control command when the position of the user's face is determined to be in the range, the bed-control command comprising an ascend command such that the first controlling unit obtains the position from the storage unit to move the headboard portion to the position based on the ascend command.

Optionally, the method further includes: triggering the detecting unit, by the first determining unit, to continue to detect the position of the user's face when the position of the user's face is determined to be not in the range.

Optionally, the bed further includes a bed-control terminal, wherein a process after the headboard portion is moved to the position includes: accepting, by the bed-control terminal, a terminal command containing an adjusted position from the user, and sending, by the bed-control terminal, the terminal command to the receiver such that the first controlling unit obtains the adjusted position from the terminal command and controls the headboard portion to move to the adjusted position.

Optionally, a process after moving the headboard portion to the adjusted position includes: outputting, by the first controlling unit, the adjusted position to the storage unit; and setting, by the storage unit, parameters of the adjusted position to be the position.

Optionally, the TV further includes a second determining unit and a detecting unit. A process before generating the bed-control command includes: detecting by the detecting unit, a user's facial state; determining, by the second determining unit, when the user is in a fatigue state or a sleeping state, and triggering, by the second determining unit, the generating unit to generate the bed-control command when the user is in the sleeping state, the bed-control command comprising a reset command such that the first controlling unit controls the headboard portion to move to a horizontal position based on the reset command.

Optionally, the TV further comprising a second controlling unit. A process after the user is determined to be in the fatigue state or in the sleeping state comprises one or more of: turning off the TV, by the second controlling unit, based on a power-off command generated by the generating unit when the second determining unit determines the user is in the sleeping state; and decreasing values of TV parameters, by the second controlling unit, based on a TV command generated by the generating unit when the second determining unit determines the user is in the fatigue state.

Optionally, the bed further includes a first connecting portion, the main portion and the headboard portion being connected through the first connecting portion. A process to move the headboard portion to the position following the bed-control command includes: moving the headboard portion about the first connecting portion to reach the position.

Optionally, the bed further includes a plurality of sub-headboard portions and a plurality of second connecting portions arranged to be parallel to the first connecting portion. A process to move a portion of the headboard portion to the position following the bed-control command includes: moving the plurality of sub-headboard portions about one or more second connecting portions until the portion of the headboard portion reaches the position.

Another aspect of the present disclosure provides a bed, including: a bed body with a main portion and a headboard portion connected to the main portion, a receiver, and a first controlling unit. The receiver is configured to receive a bed-control command sent from a TV; and the first controlling unit is configured to control the headboard portion to move to a position based on the bed-control command.

Optionally, the bed further includes a storage unit, wherein the storage unit is configured to store parameters of the position.

Optionally, the bed further includes a bed-control terminal. The bed-control terminal is configured to accept a terminal command comprising an adjusted position from the user and is configured to send the terminal command to the receiver such that the first controlling unit retrieves the adjusted position from the terminal command and controls the headboard portion to move to the adjusted position.

Optionally, the first controlling unit is configured to output the adjusted position to the storage unit; and the storage unit is configured to set parameters of the adjusted position to be parameters of the position.

Optionally, the bed further includes a first connecting portion. The main portion and the headboard portion are configured to be connected through the first connecting portion; and the headboard portion, following the bed-control command, is configured to move about the first connecting portion to reach the position.

Optionally, the bed further includes a plurality of sub-headboard portions and a plurality of second connecting portions arranged in parallel with the first connecting portion, wherein the plurality of sub-headboard portions, following the bed, are configured to move about one or more second connecting portions until a portion of the headboard portion reaches the position.

Optionally, the bed further includes a sensing unit and a sender. The sensing unit is configured to collect user movement data; the sender is configured to send the user movement data to the TV so that the TV is configured to generate a bed-control command and a TV command based on the received user movement data and send the bed-control command to the bed; the receiver is configured to receive the bed-control command and the first controlling unit is configured to adjust the headboard portion according to the bed-control command.

Another aspect of the present disclosure provides a TV, including: a generating unit and a sender. The generating unit is configured to generate a bed-control command based on a user's face position in the bed; and the sender is configured to send the bed-control command to the receiver of a bed so that the bed is configured to receive the bed-control command and control a headboard portion of the bed to move to a position based on the bed-control command.

Optionally, the TV further includes a detecting unit and a first determining unit. The detecting unit is configured to detect a position of a user's face; the first determining unit is configured to determine when the position of the user's face is in a range, and is configured to trigger the generating unit to generate the bed-control command when the position of the user's face is determined to be in the range, the bed-control command comprising an ascend command such that the bed retrieves the position from the storage unit to move the headboard portion to the position based on the ascend command.

Optionally, the first determining unit is configured to trigger the detecting unit to continue to check the position of the user's face when the position of the user's face is determined to be out of the range.

Optionally, the TV further includes a second determining unit and a detecting unit. The detecting unit is configured to detect a user's facial state; the second determining unit is configured determine when the user is in a fatigue state or a sleeping state, and is configured to trigger the generating unit to generate the bed-control command when the user is in the sleeping state, the bed-control command comprising a reset command such that the first controlling unit controls the headboard portion to move to a horizontal position based on the reset command, the reset command comprising an ascend-descend speed such that the first controlling unit controls the headboard portion to move according to the ascend-descend speed.

Optionally, the TV further includes a second controlling unit, configured to: turn off the TV based on a power-off command generated by the generating unit when the second determining unit determines the user is in the sleeping state; and decrease values of TV parameters based on a TV command generated by the generating unit when the second determining unit determines the user is in the fatigue state.

Optionally, the second determining unit is configured to determine when the user is in the fatigue state or sleeping state according to one or more fatigue detection algorithms, the one or more fatigue detection algorithms including an algorithm based on the user's eye closing time in a time period.

Optionally, the sender is configured to send the bed-control command to the bed so that the bed receives the bed-control command and adjusts the headboard portion according to the bed-control command; and the second controlling unit is configured to adjust the TV parameters according to the TV command.

### BRIEF DESCRIPTION OF THE FIGURES

The following drawings are merely examples for illustrative purposes according to various disclosed embodiments and are not intended to limit the scope of the present invention.

FIG. 1 illustrates an exemplary TV bed according to various disclosed embodiments of the present disclosure:

FIG. 2 illustrates structures of certain functional units of the TV bed illustrated in FIG. 1;

FIG. 3 illustrate certain elements used for a detection of the position of the user's face by the TV-be control system illustrated in FIG. 1;

FIG. 4 illustrates another exemplary TV bed according to various disclosed embodiments of the present disclosure;

FIG. 5 illustrates an exemplary configuration of the TV bed illustrated in FIG. 4;

FIG. 6 illustrates structures of certain functional units of the TV bed illustrated in FIG. 4:

FIG. 7 illustrates an exemplary method for operating a TV bed according to various disclosed embodiments of the present disclosure;

FIG. 8 illustrates another exemplary method for operating a TV bed according to various disclosed embodiments of the present disclosure;

FIG. 9 illustrates another exemplary method for operating a TV bed according to various disclosed embodiments of the present disclosure; and

FIG. 10 illustrates another exemplary TV bed according to various disclosed embodiments of the present disclosure.

### DETAILED DESCRIPTION

The disclosure will now describe more specifically with reference to the following embodiments. It is to be noted that the following descriptions of some embodiments are presented herein for purpose of illustration and description only. It is not intended to be exhaustive or to be limited to the precise form disclosed.

In the present disclosure, a bed refers to any type of computerized apparatus that can be used for a person to sit or lean back into a reclined position or a horizontal position. For example, a bed may be a hospital bed, a bench, a large chair, a stretcher, etc. A TV refers to any type of TV or other devices with similar image displays, such as a computer. A unit refers to a set of software program instructions and hardware components (e.g., RAM and processors) used to store and execute the software program instructions to implement described functions. A bed and a TV may thus include many functional units to collect data, store date, or transmit data.

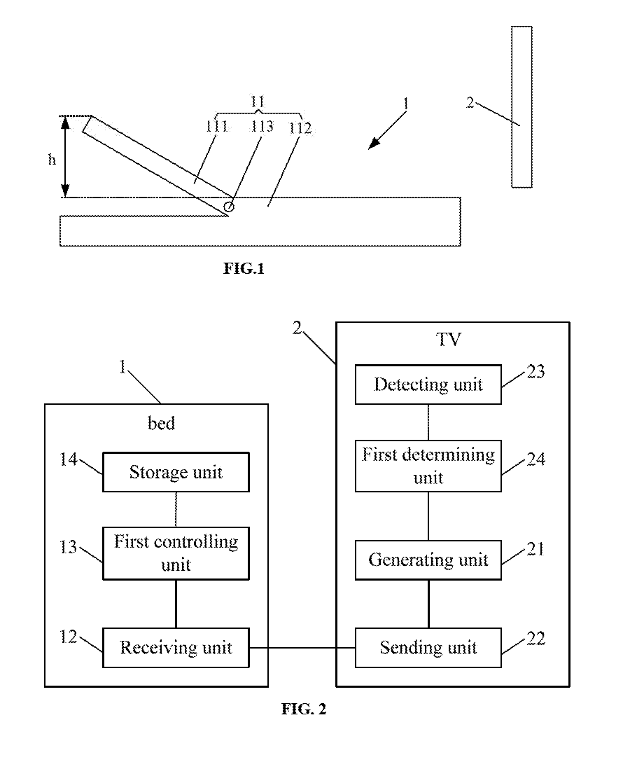

FIG. 1 illustrates the structure of an exemplary TV bed or TV-bed coordinated control system. FIG. 2 illustrates the structures of certain functional units in the TV bed depicted in FIG. 1. As shown in FIGS. 1 and 2, the TV bed may include a bed 1 and a TV 2. The bed 1 may include a bed body 11, a receiver 12, and a first controlling unit 13. The TV 2 may include a generating unit 21 and a sender 22. The bed body 11 may include a main portion (or main structure) 112 and a headboard portion (or an ascend-descend structure) 111. Specifically, in TV 2, the generating unit 21 may be connected with the sender 22. The sender 22 may be connected with the receiver 12 of bed 1. The receiver 12 may be connected with the first controlling unit 13.

The generating unit 21 may be configured to generate bed-control commands. The sender 13 may be configured to send the bed-control commands to the receiver 12. The receiver 12 may be configured to receive the bed-control commands. The first controlling unit 13 may be configured to control the headboard portion 111 of the bed body 11 to move to a preset position based on the bed-control commands or set by the bed-control commands.

In one embodiment, the sender 22 may be a wireless sender and the receiver 12 may be a wireless receiver. Thus, the data may be transmitted between the sender 22 and the receiver 12 through wireless communication means. For example, data/command signals may be transmitted through blue tooth, infrared signals, and/or Wi-Fi connections, from the sender 22 to the receiver 12.

In one embodiment, the preset position may include a preset height h, as shown in FIG. 1. The bed 1 may also include a storage unit 14, as shown in FIG. 2. The TV may also include a detecting unit 23 and a first determining unit 24. Specifically, the storage unit 14 may be connected with the first controlling unit 13. The detecting unit 23 may be connected with the first determining unit 24. The first determining unit 24 may be connected with the generating unit 21.

FIG. 3 illustrates an exemplary detection of the position of the user's face by the disclosed TV bed. As shown in FIGS. 2 and 3, the detecting unit 23 may be configured to detect the position of the user's face. The first determining unit 24 may determine if the position is in a preset range or a set region. If the first determining unit 24 determines the user's face is in the preset range, the generating unit 21 may be triggered to execute steps for generating bed-control commands. In one embodiment, the bed-control commands may include ascend commands. The detecting unit 23 may include a camera. In one embodiment, the camera may be a binocular camera. In one embodiment, the detecting unit 23 may be arranged over the TV 2 or on top of the TV 2. In one embodiment, the preset range may include a set-length region and a set-height region. The set-length region may be the region greater than L2 and less than L1. The set-height region may be the region greater than H1 and less than H2. When the user's face is located in the preset range, the position of the user's face may be described as L2<x<1 and H1<y<H2, where x represents the location of the user's face along the horizontal direction and y represents the location of the user's face along the vertical direction.

As shown in FIG. 3, H1 may represent the position, e.g., height, of the main portion 112, H2 may be a preset distance between the bottom of the TV 2 and floor, L1 may be the distance between the top of the headboard portion 111 and the TV 2, and L2 may be a preset length. Often, the positions or relative positions of the bed 1 and the TV 2 in the TV bed may not be changed once they are fixed. Thus, it may be required that the user input the values of L1, L2, H1, and H2 into the TV 2 when using the TV bed for the first time. Specifically, the values of L, L2, H1, and H2 may be inputted into the first determining unit 24. Later on, when the user turns on the TV bed each time, the detecting unit 24 may detect the position of the user's face. If the first determining unit 24 detects that the user's face is positioned in the preset range, the first determining unit 24 may determine the user is lying in bed and watching TV. Accordingly, the determining unit 24 may further trigger the generating 21 to execute subsequent steps.

Alternatively, the detecting unit 23 may be programmed to detect the bed and TV configurations during the initial calibration process of the TV bed. Instead of scanning only for a facial image, during the calibration process, the detecting unit 23 may scan the area in its scanning range, determine the position and structure of a bed, and set the values for L1, L2, H1, and H2 accordingly. The user may then be offered an opportunity to adjust the values of L1, L2, H1, and H2.

The storage unit 14 may be configured to store data such as the height h. When the TV bed is produced, the manufacturer may store a value H0 for the preset height h, where H0 is a factory default value. In other words, when the user uses the TV bed for the first time, the value of the preset height h stored in the storage unit 14 may be H0. Thus, H0 may be the default value when the TV bed starts for the first time.

The first controlling unit 13 may be configured to obtain the value of the preset height h from the storage unit 14 based on the ascend command, to control the headboard portion 111 of the bed body 11 to move up or rise to the preset height h. When the first controlling unit 13 receives the ascend command, the first controlling unit 13 may be commanded that an ascending operation may be performed to raise the headboard portion 111 of the bed body 11. Specifically, the first controlling unit 13 may control the headboard portion 111 of the bed body 11 to move from the initial position to the preset height value h. In some embodiments, the initial position may be the horizontal position.

Further, if the first determining unit 24 detects that the user's face is not in the preset range, the first determining unit 24 may trigger the detecting unit 23 to execute steps for detecting the position of the user's face. In some embodiments, if the first determining unit 24 detects the user's face is not in the preset range, the first determining unit 24 may add 1 to the detection count number and determine if the detection count number is greater than a preset count number. When the detection counter number is less than or equal to the preset counter number, the first determining unit 24 may trigger the detecting unit 23 to execute steps for detecting the position of the user's face. When the detection counter number is greater than the preset counter number, the operation may end. In another embodiment, the detection unit 23 may keep checking the detection range based on a set timer. That is, after the first check, the detection unit 23 may keep checking at a set interval until it reaches a preset length of time, e.g., for 10 minutes.

Further, the bed 1 may further include a bed-control terminal (not shown). The bed-control terminal may be configured to send terminal commands. The user may directly send terminal commands through the bed-control terminal. The receiver 12 may also be configured to receive terminal commands, which may include commands for adjusting positions, e.g., heights. The first controlling unit 13 may be configured to obtain an adjusted height H3 from the terminal commands and control the headboard portion 111 of the bed body 11 to move to the adjusted height H3. In one embodiment, when the headboard portion 111 of the bed body 11 has moved to the preset height h but and the user is not satisfied with being at the preset height h, the user may manually adjust the headboard portion 111 of the bed body 11 to move to the adjusted height H3 through the bed-control terminal.

The bed-control terminal may be, e.g., a bed remote controller, a mobile phone, and/or a tablet computer. The user may input a desired adjusted height H3 to the bed-control terminal such that the bed-control terminal may generate terminal commands accordingly. The bed-control terminal may ensure the user's control over the bed body 11 independent of the TV 2. Accordingly, it may be more flexible for the user to control the TV bed. For example, when the user, lying in bed, wants to go to bed and other people in the bedroom still want to watch TV, the user may control the headboard portion 111 to move to the horizontal position through the bed-control terminal. In this case, the user may set the adjusted height H3 to be 0 through the bed-control terminal.

Further, the first controlling unit 13 may further be configured to output the adjusted height H3 to the storage unit 14. The storage unit 14 may be configured to set the value of the preset height h to the value of the adjusted height H3. The adjusted height H3 may be the default value for the TV bed controlling system to start next time.

As shown in FIG. 1, the main portion 112 and the headboard portion 111 may be connected through a first connecting portion 113. The headboard portion 111 may follow the bed-control commands to move about the first connecting portion 113 and stop at the preset position. The movement of the headboard portion 111 may be a circular motion. In one embodiment, the first connecting portion 113 may be a first connecting shaft.

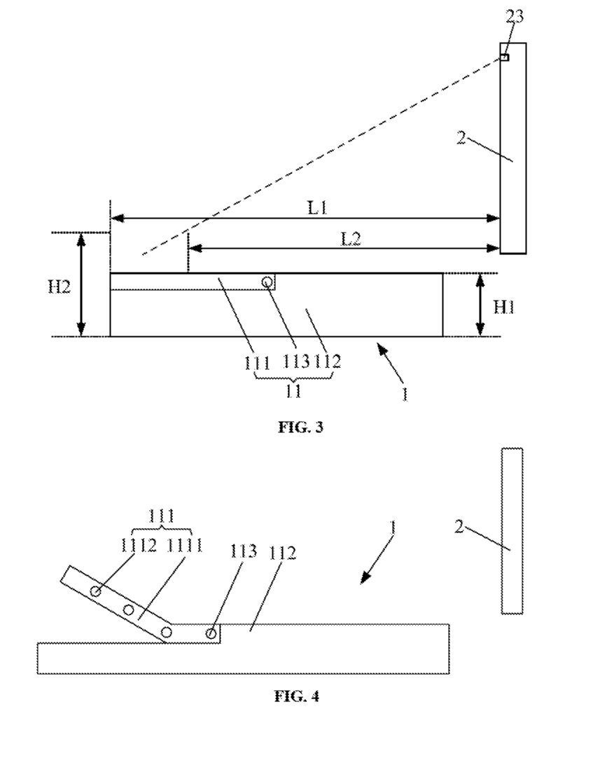

FIG. 4 illustrates another exemplary structure of the TV bed provided by the present disclosure. As shown in FIG. 4, the headboard portion 111 may include a plurality of sub-headboard portion s 1111. A sub-headboard portion 1111 may be connected with another sub-headboard portion 1111 through a second connecting portion 1112. A second connecting portion 1112 may be arranged to be parallel with the first connecting portion 113. The plurality of sub-headboard portion s 1111 may follow the bed-control commands to move around one or more second connecting portions 1112 so that a portion of the headboard portion 11 may move to the preset position. In one embodiment, the movement of the sub-headboard portion 1111 may a circular motion. The design and/or arrangement of the sub-headboard portion s 1111 and the second connecting portions 1112 may follow ergonomics. Controlled by the bed-control commands, a portion of the headboard portion 111 may move up so that this portion of the headboard portion 111 may selectively raise the body part, from the position of the cervical vertebra to the lumbar vertebra, of the user. In some embodiments, a second connecting portion 113 may be a second connecting shaft.

In one embodiment, the receiver 12, the first controlling unit 13, and the storage unit 14 may be arranged to be on the bed body 11. The generating unit 21, the sender 22, the detecting unit 23, and the first determining unit 24 may be arranged to be in the TV 2. The functional units described above are not shown in FIG. 1. In one embodiment, the storage unit 14 may be implemented in a random access memory (RAM) and/or a read only memory (ROM). The receiver 12 and the first controlling unit 13 may be implemented through the microprocessor in the bed body 11. The generating unit 21, the sender 22, the detecting unit 23, and the first determining unit 24 may be implemented through the microprocessor in the TV 2.

In embodiments of the present disclosure, the detecting unit of the TV may detect the position of the user's face, and the first determining unit may determine the user's face is positioned in the preset range to trigger the generating unit to generate an ascend command. The first controlling unit of the bed may obtain the parameters of the preset position, e.g., preset height, from the storage unit based on the ascend command and control the headboard portion of the bed to move up to the preset height. Accordingly, the user may obtain a comfortable viewing angle, and the user's cervical vertebra and lumber vertebra may be better supported. When the headboard portion of the bed body moves up to the preset height, the user may be in a reclining position. The user may be more used to the reclining position and may feel more comfortable.

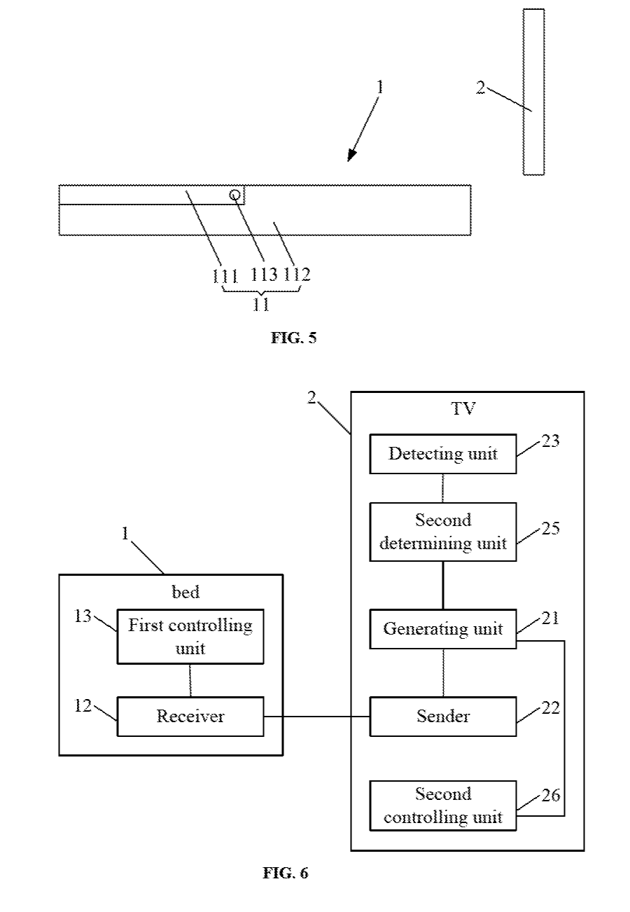

FIG. 5 illustrates the structure of the TV bed shown in FIG. 4. FIG. 6 illustrates an exemplary structure of the functional units used in the TV bed shown in FIG. 4. As shown in FIGS. 5 and 6, the TV bed may include a bed 1 and a TV 2. The bed 1 may include a bed body 11, a receiver 12, and a first controlling unit 13. The TV 2 may include a generating unit 21 and a sender 22. The bed body 11 may include a main portion 112 and a headboard portion 111 connected with the main portion 112. Specifically, the generating unit 21 and the sender 22 may be connected. The sender 22 and the receiver 12 may be connected. The receiver 12 and the first controlling unit 13 may be connected.

The generating unit 21 may be configured to generate bed-control commands. The sender 22 may be configured to send the bed-control commands to the receiver 12. The receiver 12 may be configured to receive the bed-control commands. The first controlling unit 13 may be configured to control the headboard portion 111 of the bed body 11 to move to a preset position based on the bed-control commands or set by the bed-control commands

In some embodiments, the desired position may be the initial position. The TV 2 may further include a detecting unit 23 and a second determining unit 25, as shown in FIG. 6. Specifically, the detecting unit 23 may be connected with the second determining unit 25. The second determining unit 25 may be connected with the generating unit 21.

The detecting unit 23 may be configured to detect the user's facial state. The detecting unit 23 may include a camera. In one embodiment, the camera may be a binocular camera. In one embodiment, the detecting unit 23 may be arranged over the TV 2 or on top of the TV 2. Specifically, the user's facial state may include the state of the user's eyes and/or the state of the user's mouth.

The second determining unit 25 may determine if the user is in a fatigue state and/or in a sleeping state. The second determining unit 25 may also determine if the user is not in a fatigue state or sleeping state. If the second determining unit 25 determines the user is in a sleeping state, the second determining unit 25 may trigger the generating unit 21 to execute steps for generating bed-control commands. In one embodiment, the bed-control commands may be a reset command. Specifically, based on the user's facial state, the second determining unit 25 may be configured to determine if the user is in a fatigue state and/or in a sleeping state through a fatigue detection algorithm, e.g., PERCLOS algorithm. For example, the fatigue detection algorithm may be an algorithm based on the eye closing time of the object (user). The facial state detected by the detecting unit 23 may be the eye state. The eye state may be defined to be the ratio of the total eye closing time to a set time. The second determining unit 25 may determine if the user is in a fatigue state based on the ratio. If the ratio is greater than a fatigue threshold value, the second determining unit 25 may determine the user may be in fatigue state. If the ratio is greater than a sleeping threshold, the second determining unit 25 may determine the user may be in a sleeping state. The value of the sleeping threshold may be greater than the value of the fatigue threshold. In practice, other suitable algorithms may also be used to determine if the user is in fatigue state of in sleeping state. In certain embodiments, the changes in the user's eye shape and/or mouth shape may be recorded and a machine learning process may be applied to determine if the user is in fatigue state. In certain other embodiments, the sizes of pupils and the times of blink in a certain time may be recorded and compared to references that represent the user's sizes of pupils and times of blink at normal circumstances, to determine if the user is in fatigue state. The specific algorithm to determine the user's facial state should be determined according to different applications and/or designs and should not be limited by the embodiments of the present disclosure.

The first controlling unit 13 may be configured to control the headboard portion 111 of the bed body 11 to move to the initial position based on the reset command. In one embodiment, the initial position may be the horizontal position. When the first controlling unit receive the reset command, the first controlling unit 13 may be commanded to perform a reset operation on the headboard portion 111 of the bed body 11. In one embodiment, the reset command may include an ascend-descend speed. That is, the first controlling unit 13 may control the headboard portion 111 of the bed body 11 to move to the initial position at the ascend-descend speed, based on the reset command. The generating unit 21 may generate the ascend-descend speed, and generate the reset command based on the ascend-descend speed. Also, the ascend-descend speed may be stored in the storage module in the TV 2. The generating unit 21 may obtain the ascend-descend speed from the storage module in the TV 2 and generate the reset command based on the ascend-descend speed. The first controlling unit 13 may control the headboard portion 111 of the bed body 11 to move to the initial position at the ascend-descend speed. The user can have an improved comfort level when sleeping.

Further, the TV 2 may further include a second controlling unit 26. Specifically, the second controlling unit 26 may be connected to the generating unit 21.

The generating unit 21 may be configured to generate a power-off command if the second determining unit 25 detects the user is in a sleeping state. The second controlling unit 26 may be configured to turn off the TV 2 based on the power-off command.

The generating unit 21 may generate TV commands if the second determining unit 25 determines the user is in a fatigue state. The second controlling unit 26 may decrease the values of the TV parameters based on the TV commands. For example, the TV commands may include the commands for decreasing the volume and/or decreasing the brightness, of the display of TV 2. When the TV commands include commands for decreasing the volume of the TV 2, the TV parameters may include volume parameters. When the TV commands include commands for reducing the brightness of the TV 2, the TV parameters may include volume parameters. In practice, the TV commands may also be other suitable commands for controlling the TV 2, and should not be limited by the embodiments of the present disclosure. In some embodiments, the generating unit 21 may generate TV commands directly. In some other embodiments, commands, e.g., for decreasing the volume and/or decreasing the brightness of the TV 2, may be stored in the storage module in the TV 2. The generating unit 21 may obtain desired commands, e.g., commands for decreasing the volume and/or decreasing the brightness of the TV 2, from the storage module, and generate TV commands based on the obtained commands.

The generating unit 21 may also be configured to trigger the detecting unit 23 for detecting the user's facial state if the second determining unit 25 determines the user is not in a fatigue state or in a sleeping state.

The structure and operation of the headboard portion 111 may be referred to the description of the TV bed shown in FIGS. 1-3 and are not repeated herein.

In one embodiment, the receiver 12 and the first controlling unit 13 may be arranged on the bed body 11. The generating unit 21, the sender 22, the detecting unit 23, the second determining unit 25, and the second controlling unit 26 may be arranged over or on top of the TV 2. The abovementioned functional units are not shown in FIG. 4. In one embodiment, the receiver 12 and the first controlling unit 13 may be implemented through the microprocessor on the bed body 11. The generating unit 21, the sender 22, the detecting unit 23, the second determining unit 25, and the second controlling unit 26 may be implemented through the microprocessor in the TV 2.

In embodiments of the present disclosure, the detecting unit of the TV may detect the user's facial state, the second determining unit may determine if the user is in a sleeping state. When the user is in a sleeping state, the generating unit may generate a reset command. The first controlling unit of the bed may control the headboard portion to move to the initial position based on the reset command so that the user may obtain a more comfortable sleeping angle. The user's cervical vertebra and lumbar vertebra may be better protected. In one embodiment, when detecting the user is in a sleeping state, the disclosed TV bed may control the headboard portion of the bed body to move to the horizontal position, and the user may have improved quality of sleep. In one embodiment, when the user is detected to be in a sleeping state, the disclosed TV bed may turn off the TV, and the user's quality of sleep may be further improved. In one embodiment, when the user is detected to be in a fatigue state, the TV bed may decrease the values of the parameters of the TV to produce a hypnotic effect on the user. Thus, the TV bed may help the user fall asleep.

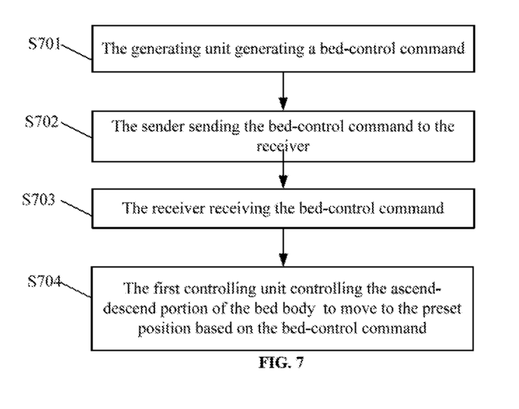

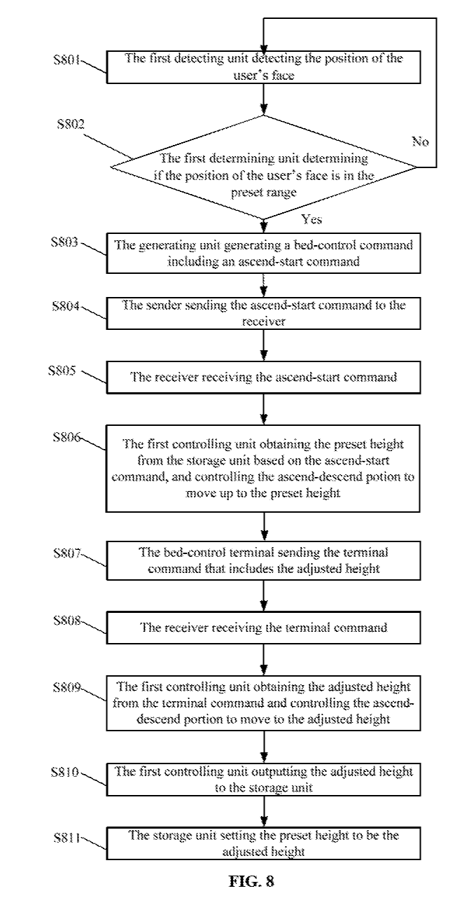

FIG. 7 illustrates a flow chart of an exemplary method for operating the TV bed according to the embodiments of the present disclosure. The TV bed may include a bed and a TV. The bed may include a bed body, a receiver, and a first controlling unit. The TV may include a generating unit and a sender. The bed body may include a main portion and a headboard portion connected to the main portion.

As shown in FIG. 7, the disclosed method may include steps S701-S704.

In step S701, the generating unit may generate a bed-control command.

In step S702, the sender may send the bed-control command to the receiver.

In step S703, the receiver may receive the bed-control command.

In step S704, the first controlling unit may control the headboard portion of the bed body to move to a preset position based on the bed-control command.

In embodiments of the present disclosure, the generating unit of the TV may generate a bed-control command, and the first controlling unit of the bed may control the headboard portion of the bed body to move to the preset position based on the bed-control command. The user may be more comfortable when watching TV, and the user's cervical vertebra and lumbar vertebra may be better supported.

FIG. 8 illustrates a process flow of another exemplary method for operating the TV bed. The TV bed may include a bed and a TV. The bed may include a bed body, a receiver, a first controlling unit, a storage unit, and a bed-control terminal. The TV may include a generating unit, a sender, a detecting unit, and a first determining unit. The bed body may include a main portion and a headboard portion connected to the main portion.

As shown in FIG. 8, the method may include steps S801-S811.

In step S801, the detecting unit may detect the position of the user's face.

In step S802, the first determining unit may determine if the user's face is in a preset range. If the first determining unit determines the user's face is in the preset range, the process may proceed to step S803. If the first determining unit determines the user's face is not in the preset range, the process may return to step S801 and continue to proceed from step S801.

In step S803, the generating unit may generate a bed-control command. The bed-control command may be an ascend command.

In step S804, the sender may send the ascend command to the receiver.

In step S805, the receiver may receive the ascend command.

In step S806, the first controlling unit may obtain parameters of a preset height from the storage module based on the ascend command.

In step S807, the bed-control terminal may send a terminal command. The terminal command may include parameters of an adjusted height.

In step S808, the receiver may receive the terminal command.

In step S809, the first controlling unit may obtain the adjusted height from the terminal command and control the headboard portion of the bed body to move to the adjusted height.

In step S810, the first controlling unit may output the adjusted height to the storage unit.

In step S811, the storage unit may set the parameters of the preset height to be the parameters of the adjusted height.

In embodiments of the present disclosure, the detecting unit of the TV may detect the position of the user's face, and the first determining unit may detect if the user's face is positioned in the preset range. When the user's face is in the preset range, the generating unit may generate the ascend command. The first controlling unit of the bed may obtain the preset height from the storage unit based on the ascend command, and control the headboard portion of the bed body to move up to the preset height. Accordingly, the user may have a more comfortable viewing angle, and the user's cervical vertebra and lumbar vertebra may be better supported. When the headboard portion of the bed body move up to the preset height, the user may be in a reclining position. The user may be more used to the reclining position and may feel more comfortable.

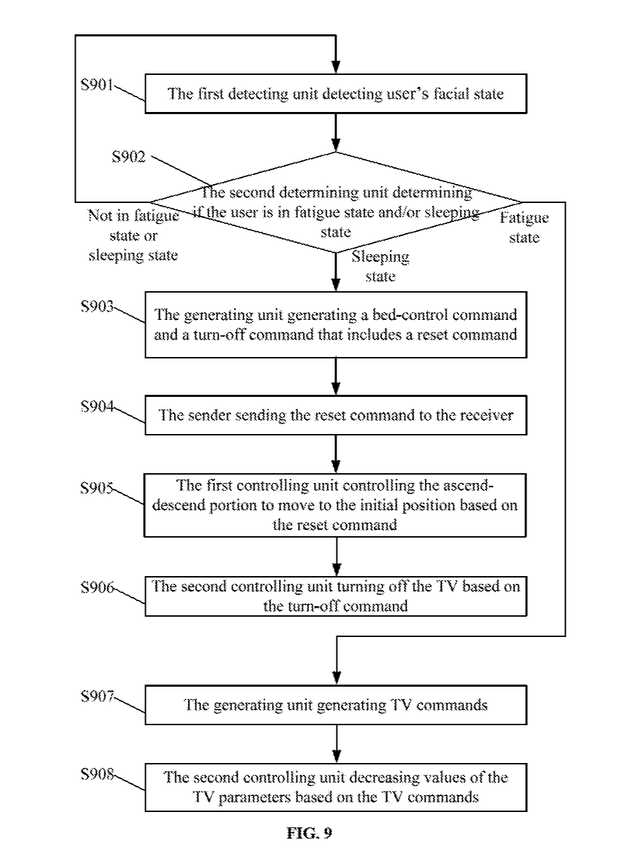

FIG. 9 illustrates a process flow of another exemplary method for operating the TV bed. The TV bed may include a bed and a TV. The bed may include a bed body, a receiver, and a first controlling unit. The TV may include a generating unit, a sender, a detecting unit, a second determining unit, and a second controlling unit. The bed body may include a main portion and a headboard portion connected to the main portion. The default position may be the initial position.

As shown in FIG. 9, the disclosed method may include steps S901-S908.

In step S901, the detecting unit may detect the user's facial state.

In step S902, the second determining unit may determine if the user is in a fatigue state and/or a sleeping state, or if the user is not in a fatigue state or a sleeping state, based on the user's facial state. If the second determining unit determines that the user is in a sleeping state, the process may proceed to step S903. If the second determining unit determines the user is in a fatigue state, the process may proceed to step S907. If the second determining unit determines the user is not in a fatigue state or a sleeping state, the process may return to step S901 and proceed from step S901.

In step S903, the generating unit may generate a bed-control command and a power-off command. The bed-control command may include a reset command.

In step S904, the sender may send the reset command to the receiver.

In step S905, the first controlling unit may control the headboard portion of the bed body to move to the initial position based on the reset command.

In step S906, the second controlling unit may turn off the TV based on the power-off command. The process may end.

In step S907, the generating unit may generate TV commands.

In step S908, the second controlling unit may decrease the values of certain parameters of the TV based on the TV commands. The process may end.

In the embodiments provided in the present disclosure, the order of the operational steps is executed may only be exemplary. In practice, the order of the operational steps may be flexibly adjusted according to different applications and/designs. For examples, in one embodiment, step S906 may be executed first, and steps S904 and S905 may be executed later. In another embodiment, steps S906, S904, and S905 may be executed at the same time.

In certain embodiments, the user may modify or set the preset position, the preset height, and the initial position manually through the bed-control terminal or directly on the TV or on the bed. That is, in certain embodiments, the bed and the TV may include certain interfaces, e.g., touch screens and/or buttons, for the user to type in desired parameters. The interfaces may be connected to the microprocessors of the TV and/or the bed so that the user's command may be processed to modify or overwrite certain factory default values and values recorded in the TV bed.

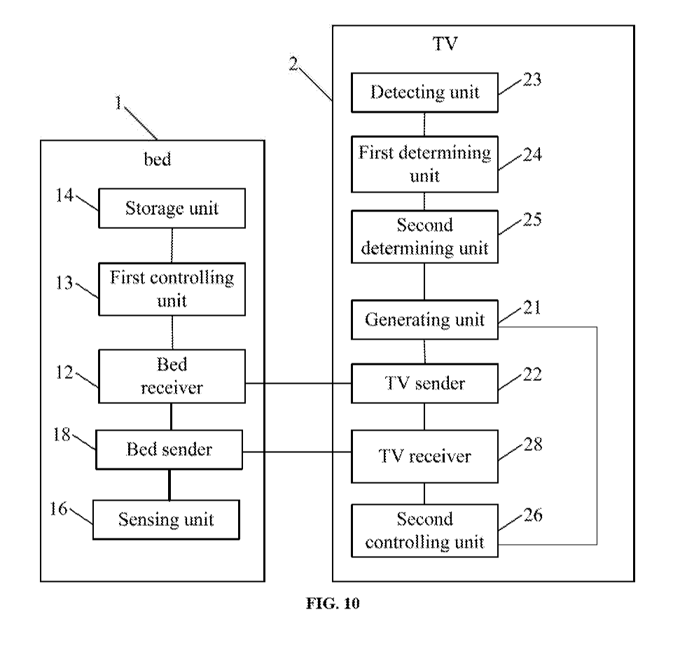

In some embodiments of the present disclosure, the TV bed may be set on a timer to run a going-to-sleep routine. For example, the detecting unit 23 may start detecting the position of a user's face at a pre-determined time of day, e.g., at 22:00 daily. Alternatively, the detecting unit 23 may be set on a weekly or monthly schedule so that it can run the going-to-sleep routine on the set schedule. An exemplary TV bed is illustrated in FIG. 10. As shown in FIG. 10, the bed 1 may include a storage unit 14, a first controlling unit 13, a bed receiver 12, a bed sender 18, and a sensing unit 16. The TV 2 may include a detecting unit 23, a first determining unit 24, a second determining unit 25, a generating unit 21, a TV sender 22, a TV receiver 28, and a second controlling unit 26. The TV sender 22 and the bed receiver 12 may communicate through wireless means. The bed sender 18 and the TV receiver 28 may communicate through wireless means, the bed receiver 12 may be configured to receive command signals from the TV sender 22, and the TV receiver 28 may be configured to receive command signals from the bed sender 18.

Exemplary steps of the going-to-sleep routine are described below.

At the pre-determined time, e.g., at 22:00 at night, the detecting unit 23 may detect the position of the user's face. This is the preset time for the user to go to bed.

Further, the first determining unit may determine if the user's face is in a preset range. If the first determining unit determines the user's face is in the preset range, the process may proceed to generate a bed-control command. If the first determining unit determines the user's face is not in the preset range, the process may return to the previous step and continue to check the position of the user's face.

Further, once the user's face is in the preset area, the generating unit may generate a bed-control command. The bed-control command may be an ascend command. The TV sender 22 may send the ascend command to the bed receiver 12. The bed receiver 121 may receive the ascend command. Further, the first controlling unit 13 may obtain a preset height from the storage unit 14 based on the ascend command. The headboard portion of the bed 1 may be moved up to the preset height

Further, after the user's face is detected, a set time (e.g., 5 minutes) after the detection, the detecting unit 23 may detect the user's facial state. Further, the second determining unit 26 may determine if the user is in a fatigue state and/or a sleeping state, or if the user is not in a fatigue state or a sleeping state, based on the user's facial state. Further, if the second determining unit determines that the user is in a sleeping state, the process may proceed to generate a power-off command to send to the bed receiver 12. The power-off command may include an information to reset the bed 1 to a flat/initial position. Further, the first controlling unit 13 may control the headboard portion of the bed body to move to the initial position based on the reset command. Further, the second controlling unit 26 may turn off the TV based on the power-off command. The process may end.

Further, if the second determining unit determines the user is in a fatigue state, the generating unit 21 may generate TV commands to turn down the volume and lower the brightness level of the TV display. Further, the second controlling unit 26 may turn down the volume and lower the brightness level of the TV display according to the TV commands.

If the second determining unit 25 determines the user is not in a fatigue state or a sleeping state, the process may go back to detecting the user's facial state, checking the user's fatigue state or sleeping state again.

In embodiments of the present disclosure, at a pre-determined bedtime, the detecting unit of the TV may detect the position of the user's face, and the first determining unit may detect if the user's face is positioned in the preset range. When the user's face is in the preset range, the generating unit may generate the ascend command. The first controlling unit of the bed may obtain the preset height from the storage unit based on the ascend command, and control the headboard portion of the bed body to move up to the preset height. Next, the detecting unit may determine whether the user is in a fatigue state or a sleeping state. If the user is in a sleeping state, the generating unit may generate bed-control bed to power-off and reset the bed position. The second controlling unit may turn off the TV. If the user is in a fatigue state, the generating unit may generate bed-control bed to lower the headboard portion of the bed. The second controlling unit may set the TV to a lower volume and dimmer brightness level. If the user is not in a fatigue or sleeping state, the detecting unit keeps checking the state of the user at a set interval, e.g., every 3 minutes.

As shown in FIG. 10, in some embodiments, the bed in the TV bed may include a sensing unit 16 that collects sensing data, such as pressure, temperature, etc. of the user in bed 1. The sensing unit may include one or more temperature sensors, pressure sensors, etc. The bed may also include a sender 18 that sends data to the TV. The TV may include a receiver 28 that receives data from the bed 1.

In some embodiments of the present disclosure, the TV bed may implement a wake-up routine. The wake-up routine may be set to ran based on a set timer, such as every morning at 7:00 o'clock. Alternatively, the wake-up routine may be set to run a number of hours after the going-to-sleep routine was run. In one example, the detecting unit of the TV may scan the user's face at the preset time (e.g., 7:00 in the morning). Based on the user's face movements (e.g., eyelid movements or eye opening or closing), the determining unit of the TV may determine whether to start the wake-up routine. In another example, the wake-up may be set up to run eight hours after the going-to-sleep routine ended. Further, instead of setting up the wake-up routine using a timer, the bed 1 may use the sensing unit 16 to measure the movements of the user (e.g., user sitting up, lean forward, etc.), and send the sensing data to TV 2 through the sender 18. Based on the sensing data, the first determining unit 24 and the second determining unit 25 may determine whether to start the wake-up routine.

In some embodiments, the generating unit 21 may generate a bed-control command and a turn-on command. The bed-control command may be used to lift up the headboard portion to a preset wake-up height. Further, the TV sender 22 may send the bed-control command to the bed receiver 12. Further, the bed receiver 12 may receive the bed-control command. Further, the first controlling unit 13 may set the headboard portion of the bed to the wake-up height. Further, the second controlling unit 26 may turn on the TV and the audio signal to the TV, as a part of an improved user wake-up experience.

In the present disclosure, the components, e.g., units, of the TV bed may be implemented by any appropriately configured computer system. Such a computer system often includes a processor, a random access memory (RAM), a read-only memory (ROM), a storage, a display, an input/output interface, a database, and a communication interface. Other components may be added and certain devices may be removed without departing from the principles of the disclosed embodiments.

The processor may include any appropriate type of general purpose microprocessor, digital signal processor or microcontroller, and application specific integrated circuit (ASIC). Processor may execute sequences of computer program instructions to perform various processes associated with control portion. Computer program instructions may be loaded into RAM for execution by the processor from read-only memory, or from storage. Storage may include any appropriate type of mass storage provided to store any type of information that the processor may need to perform the disclosed processes. For example, storage may include one or more hard disk devices, optical disk devices, flash disks, or other storage devices to provide storage space. In some embodiments, the processor may include the microprocessors distributed in the bed and the TV.

The Input/output interface may be provided for users to input information into the TV bed or for the users to receive information from the TV bed. For example, input/output interface may include any appropriate input device, such as a keyboard, a mouse, an electronic tablet, voice communication devices, touch screens, or any other optical or wireless input devices. Further, input/output interface may receive from and/or send to other external devices, such as a bed-control terminal.

Further, the database may include any type of commercial or customized database, and may also include analysis tools for analyzing the information in the databases. Database may be used for storing, e.g., certain algorithms and other related information. For example, a database may be used to store the fatigue detection algorithm and a database may be used to store various bed position or headboard portion heights. Communication interface may provide communication connections such that control portion may be accessed remotely and/or communicate with other systems through computer networks or other communication networks via various communication protocols, such as transmission control protocol/internet protocol (TCP/IP), hypertext transfer protocol (HTTP), etc.

In one embodiment, the processor may obtain values of the preset height, L1, L2, H1, and H2 stored in the database or storage, obtain the user's facial movement from the communication interface through the camera, and obtain a fatigue detection algorithm from the database to determine if the user's face is positioned in the preset range. If the user's face is in the preset range, the processor may generate a bed-control command that includes an ascend command. The ascend command may be transmitted to the communication interface and further transmitted to the electrical parts and mechanical parts included in the headboard portion of the bed body to move the headboard portion up to the preset height.

In embodiments of the present disclosure, the detecting unit of the TV may detect the user's facial state, the second determining unit may determine if the user is in a sleeping state. When the user is in a sleeping state, the generating unit may generate a reset command. The first controlling unit of the bed may control the headboard portion to move to the initial position based on the reset command so that the user may obtain a more comfortable sleeping angle. The user's cervical vertebra and lumbar vertebra may be better supported.

In one embodiment, when detecting the user is in a sleeping state, the disclosed TV bed may control the headboard portion of the bed body to move back to the horizontal position, and the user may have improved quality of sleep. In one embodiment, when detecting the user is in a sleeping state, the disclosed TV bed may turn off the TV, and the user's quality of sleep may be further improved. In one embodiment, when detecting the user is in a fatigue state, the TV bed may decrease the values of the parameters of the TV to produce a hypnotic effect on the user. Thus, the TV bed may help the user fall asleep. Further, when detecting the user is waking up, the disclosed TV bed may control the headboard portion of the bed body to lift up the person's torso. The TV bed may turn on the TV display. Thus, the TV bed may help the user wake up.

For illustrative purposes, the TV bed in the present disclosure is illustrated as a whole piece. However, the TV and the bed in the TV bed may be controlled and used separately. That is, TV and the bed may each respond to external signals sent from other devices to each perform the functions described in the embodiments. For example, a control application may be installed on a smart phone, and the user may control the TV to adjust TV parameters separately from bed, and may also control the bed to adjust height separately from the TV. Details to control the TV and the bed may be referred to previous description of the disclosed TV bed and are not repeated herein.

The foregoing description of the embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, the reference to exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

1. A TV bed, comprising:
a bed and a TV, wherein the bed comprises a bed body with a main portion and a headboard portion, a bed sensing unit, a bed receiver, a bed sender and a first controlling unit; and the TV comprises a detecting unit, a determining unit, a generating unit, a TV receiver, a TV sender, and a second controlling unit, the bed receiver being configured to receive command signals from the TV sender, and the TV receiver being configured to receive command signals from the bed sender, wherein:
the TV is configured to generate a bed-control command based on a user's face position in the bed and is configured to send the bed-control command to the bed; and
the bed is configured to receive the bed-control command and is configured to control the headboard portion to move to a position based on the bed-control command.

2. The TV bed according to claim 1, wherein:
the TV is configured to determine whether the user is in a fatigue state, a sleeping state, or a wake-up state and is configured to generate a bed-control command and a TV command according to a result of determination;
the bed is configured to receive the bed control-command and is configured to control the headboard portion according to the bed-control command; and
the TV is configured to adjust TV parameters according to the TV command.

3. The TV bed according to claim 2, wherein:
the TV is configured to determine whether the user is in the fatigue state, the sleeping state, or the wake-up state by scanning the user's face.

4. The TV bed according to claim 2, wherein:
the bed is configured to measure user data and sends the user data to the TV; and
the TV is configured to determine whether the user is in the fatigue state, the sleeping state, or the wake-up state based on the user data.

5. A TV bed, comprising:
a bed and a TV, wherein the bed is configured to comprise a bed body with a main portion and a headboard portion connected to the main portion, a receiver, and a first controlling unit; and the TV comprises a generating unit and a sender, the receiver, wherein:
the generating unit is configured to generate a bed-control command based on a user's face position in the bed;
the sender is configured to send the bed-control command to the receiver of the bed;
the receiver is configured to receive the bed-control command; and
the first controlling unit is configured to control the headboard portion to move to a position based on the bed-control command.

6. The TV bed according to claim 5, the bed further comprising a storage unit, the TV further comprising a detecting unit and a first determining unit, wherein:
the storage unit is configured to store parameters of the position;
the detecting unit is configured to detect a position of a user's face; and
the first determining unit is configured to determine when the position of the user's face is in a range, and is configured to trigger the generating unit to generate the bed-control command when the position of the user's face is determined to be in the range, the bed-control command comprising an ascend command such that the first controlling unit retrieves the position from the storage unit to move the headboard portion to the position based on the ascend command.

7. The TV bed according to claim 6, wherein the first determining unit is configured to trigger the detecting unit to continue to check the position of the user's face when the position of the user's face is determined to be out of the range.

8. The TV bed according to claim 6, the bed further comprising a bed-control terminal, wherein:
the bed-control terminal is configured to accept a terminal command comprising an adjusted position from the user and is configured to send the terminal command to the receiver of the bed such that the first controlling unit retrieves the adjusted position from the terminal command and controls the headboard portion to move to the adjusted position.

9. The TV bed according to claim 8, wherein:
the first controlling unit is configured to output the adjusted position to the storage unit; and
the storage unit is configured to set parameters of the adjusted position to be parameters of the position.

10. The TV bed according to claim 5, the TV further comprising a second determining unit and a detecting unit,
wherein:
the detecting unit is configured to detect a user's facial state; and
the second determining unit is configured determine when the user is in a fatigue state or a sleeping state, and is configured to trigger the generating unit to generate the bed-control command when the user is in the sleeping state, the bed-control command comprising a reset command such that the first controlling unit controls the headboard portion to move to a horizontal position based on the reset command, the reset command comprising an ascend-descend speed such that the first controlling unit controls the headboard portion to move according to the ascend-descend speed.

11. The TV bed according to claim 10, the TV further comprising a second controlling unit, wherein the second controlling unit is configured to:
turn off the TV based on a power-off command generated by the generating unit when the second determining unit determines the user is in the sleeping state; and
decrease values of TV parameters based on a TV command generated by the generating unit when the second determining unit determines the user is in the fatigue state.

12. The TV bed according to claim 10, wherein the second determining unit is configured to determine when the user is in the fatigue state or sleeping state according to one or more fatigue detection algorithms, the one or more fatigue detection algorithms including an algorithm based on the user's eye closing time in a time period.

13. The TV bed according to claim 5, the bed further comprising a first connecting portion, wherein:
the main portion and the headboard portion are configured to be connected through the first connecting portion; and
the headboard portion, following the bed-control command, is configured to move about the first connecting portion to reach the position.

14. The TV bed according to claim 13, the bed further comprising a plurality of sub-headboard portions and a plurality of second connecting portions arranged in parallel with the first connecting portion, wherein the plurality of sub-headboard portions, following the bed, are configured to move about one or more second connecting portions until a portion of the headboard portion reaches the position.

15. The TV bed according to claim 5, the bed further comprising a sensing unit and a sender and the TV further comprising a receiver wherein:
the sensing unit is configured to collect user movement data;
the sender of the bed is configured to send the user movement data to the receiver of the TV; and
the generating unit is configured to generate a bed-control command and a TV command based on the received user movement data.

16. The TV bed according to claim 15, wherein:
the sender of the TV is configured to send the bed-control command to the bed;
the receiver is configured to receive the bed-control command;
the first controlling unit is configured to adjust the headboard portion according to the bed-control command; and
the second controlling unit is configured to adjust the TV parameters according to the TV command.

17. A method for controlling a TV bed, the TV bed comprising a bed and a TV, wherein the bed comprises a bed body, a receiver, and a first controlling unit; and the TV comprises a generating unit and a sender, the bed body comprising a main portion and a headboard portion connected to the main portion, the method comprising:
generating, by the generating unit, a bed-control command based on a user's face position in the bed;
sending, by the sender of the TV, the bed-control command to the receiver of the bed;
receiving, by the receiver of the bed, the bed-control command; and
controlling, by the first controlling unit, the headboard portion of the bed body to move to a position based on the bed-control command.

18. The method according to claim 17, the bed further comprising a storage unit, the TV further comprising a detecting unit and a first determining unit, wherein a process before generating the bed-control command comprises:
storing, by the storage unit, parameters of the position;
detecting, by the detecting unit, a position of a user's face; and
determining, by the first determining unit, when the position of the user's face is in a range, and triggering, by the first determining unit, the generating unit to generate the bed-control command when the position of the user's face is determined to be in the range, the bed-control command comprising an ascend command such that the first controlling unit obtains the position from the storage unit to move the headboard portion to the position based on the ascend command.

19. The method according to claim 18, further comprising: triggering the detecting unit, by the first determining unit, to continue to detect the position of the user's face when the position of the user's face is determined to be not in the range.

20. The method according to claim 18, the bed further comprising a bed-control terminal, wherein a process after the headboard portion is moved to the position comprises:
accepting, by the bed-control terminal, a terminal command containing an adjusted position from the user; and
sending, by the bed-control terminal, the terminal command to the receiver such that the first controlling unit obtains the adjusted position from the terminal command and controls the headboard portion to move to the adjusted position.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)
